(19) United States
(12) Patent Application Publication
(10) Pub. No.: US 2026/0325817 A1
(43) Pub. Date: Oct. 22, 2026

(54) BALL VALVE FOR USE IN A RESPIRATION CIRCUIT AND A RESPIRATION CIRCUIT INCLUDING A BALL VALVE

(71) Applicant: Engineered Medical Systems, Inc., Indianapolis, IN (US)

(72) Inventors: Bradley Allen Wheeler, Martinsville, IN (US); Jessica Mae Zinnecker, Indianapolis, IN (US); Kevin Michael Sempsrott, Anderson, IN (US)

(21) Appl. No.: 19/633,063

(22) Filed: Mar. 30, 2026

Related U.S. Application Data
(63) Continuation of application No. 17/483,374, filed on Sep. 23, 2021, now Pat. No. 12,589,233.

(51) Int. Cl. A61M 39/24; A61M 16/06; A61M 39/22; F16K 11/087

(52) U.S. Cl. CPC A61M 39/24; A61M 16/06; A61M 39/223; F16K 11/0873; A61M 39/229; A61M 2039/248

(57) ABSTRACT

A ball valve for use in a respiration circuit proving for selective gas flow between at least two outlets of the ball valve and a respiration circuit including the ball valve.

### CROSS REFERENCE TO RELATED APPLICATION

This application is a continuation of U.S. patent application Ser. No. 17/483,374 filed Sep. 23, 2021, the entire disclosure of which is hereby incorporated by reference.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

### TECHNICAL FIELD

The present disclosure is directed to a respiration circuit, and more particularly to a ball valve for use in a respiration circuit and a respiration circuit including a ball valve.

### BACKGROUND

Respiration circuits are used in a variety of respiratory therapies. In some instances, respiratory therapies require a pressurized gas supply to two or more devices. For example, when a patient is receiving CPAP, particularly on an ongoing basis, humidifying the pressurized gas delivered to the patient by means of a nebulizer can increase patient comfort and help protect sensitive tissue from drying out. For other therapies, a nebulizer can be useful for administering nebulized medications to a patient receiving respiratory support. These therapies typically require two separate sources of pressurized gas be provided, or a Y-splitter from a single source be provided in the respiration circuit. Providing two sources of pressurized gas can be difficult in a home setting and complicates the circuit. A Y-splitter has the limitation that if the gas supply needs to be terminated in one but not both of the devices, the gas supply must be cut off to both at least temporarily to remove the Y-splitter, which is awkward, time consuming and can prove uncomfortable or even harmful to the patient.

The present invention is directed toward overcoming one or more of the problems discussed above.

### SUMMARY OF THE EMBODIMENTS

A first aspect of the disclosure is a ball valve for a use in a respiration circuit. In one embodiment the ball valve includes a housing defining a ball chamber, the ball chamber communicating with a gas inlet and at least two gas outlets. A ball is received in the ball chamber, the ball being configured for rotation about an axis of rotation within the ball chamber. The ball has a channel extending perpendicular to the axis of rotation, the channel terminating in a channel inlet configured for fluid communication with the gas inlet. The channel inlet is in fluid communication with a channel outlet configured for selective fluid communication with each of the at least two gas outlets. An actuator is configured to rotate the ball about the axis of rotation. The actuator can rotate the ball to a first position with the gas inlet in fluid communication with the channel inlet and the channel outlet in fluid communication with each of the at least two gas outlets, a second position with the gas inlet in fluid communication with the channel inlet and the gas outlet in fluid communication with only one gas outlet of the at least two gas outlets, and a third position with the gas inlet in fluid communication with the channel inlet and the gas outlet in fluid communication with at least one other gas outlet of the at least two gas outlets.

Various embodiments can be limited to two gas outlets. In such embodiments each of the two outlets can have different sized cross-sections to facilitate different rates of flow volumes through the respective gas outlets. Various embodiments can include the actuator being configured to rotate the ball to a fourth position with the gas inlet not in fluid communication with the channel inlet. In various embodiments, the actuator comprises a stem extending from the ball along the axis of rotation and a handle operatively associated with a distal end of the stem.

Various embodiments can include a first elastomeric O-ring surrounding the gas inlet between a surface of the ball chamber and a surface of the ball, a second elastomeric O-ring surrounding one of the at least two gas outlets between the surface of the ball chamber and the surface of the ball, a third elastomeric O-ring surrounding at least one other of the at least two gas outlets between the surface of the ball chamber and the surface of the ball. In such embodiments in the first position the channel inlet is at least partially within a circumference of the first O-ring and the channel outlet is at least partially within the a circumference of the second O-ring and the third O-ring; in the second position the channel inlet is at least partially within the circumference of the first O-ring and the channel outlet is at least partially within the circumference of the second O-ring and the channel outlet is completely outside the circumference of the third O-ring; and in the third position the channel inlet is at least partially within the circumference of the first O-ring and the channel outlet is at least partially within the circumference of the third O-ring and channel outlet is completely outside the circumference of the second O-ring. In embodiments including a fourth position of the actuator, the channel inlet is completely outside the circumference of the first O-ring. In various embodiments the channel is elliptical with the major axis perpendicular to the axis of rotation and the major axis in channel inlet being shorter than the major axis in the channel outlet. Embodiments can also include a fourth elastomeric O-ring surrounding the first elastomeric O-ring and the gas inlet between a surface of the ball chamber and a surface of the ball positioned to prevent gas flow between the ball surface and a ball chamber surface outside the circumference of the fourth elastomeric O-ring with the ball in any of the first, second, third or fourth positions and a fifth elastomeric O-ring surrounding the second and third elastomeric O-rings and each of the at least two gas outlets between the surface of the ball chamber and the surface of the ball positioned to prevent gas flow between the ball surface and the ball chamber surface outside the circumference of the fifth elastomeric O-ring with the ball in any of the first, second, third or fourth positions.

Another aspect of the invention is a respiration circuit system including a ball valve incorporating one or more of the various embodiments discussed above. The respiration circuit system further includes a mask having a mask inlet, the mask inlet being in fluid communication with the one gas outlet of the at least two gas outlets and a nebulizer having nebulizer inlet and a nebulizer outlet, the nebulizer inlet being in fluid communication with the at least one other gas outlet of the at least two gas outlets and the nebulizer outlet being in fluid communication with the mask.

Various modifications and additions can be made to the embodiments discussed without departing from the scope of the invention. For example, while the embodiments described above refer to particular features, the scope of this invention also included embodiments having different combination of features and embodiments that do not include all of the above-described features.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. In some instances, a sub-label is associated with a reference numeral to denote one of multiple similar components. When reference is made to a reference numeral without specification to an existing sub-label, it is intended to refer to all such multiple similar components.

FIG. 1 is a perspective view of ball valve for use in a respiration circuit in accordance with the present disclosure with a threaded connector attached to an gas inlet of the ball valve;

FIG. 2 is an exploded view of the ball valve and quick-connect adapter of FIG. 1;

FIG. 3 is a perspective view of the ball valve with a threaded connector attached to an gas inlet of FIG. 1 further including a quick-connect fitting attached to the threaded connector;

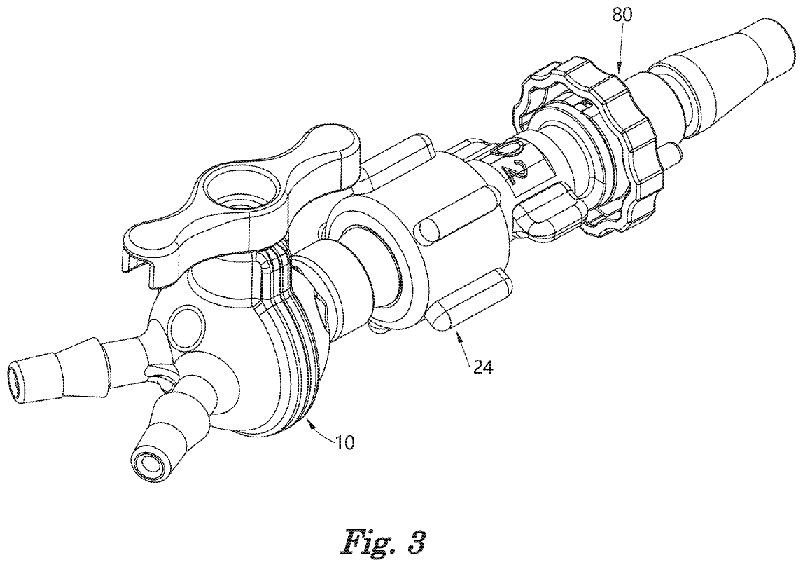

FIG. 4 is an exploded view of the assembly of FIG. 3;

FIGS. 5A-5D are cross-sectional views of the ball valve of FIG. 1 taken along line 5-5 of FIG. 1 showing the ball in first, second, third and fourth positions, respectively;

FIG. 6 is a cross-sectional view of the ball valve of FIG. 1 taken along line 6-6 of FIG. 1 showing the ball in the first position;

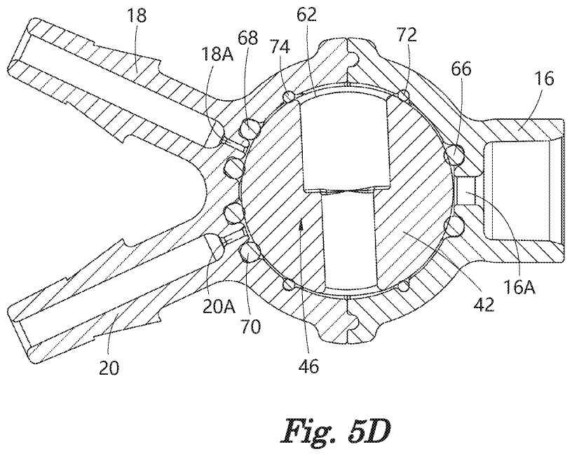

FIG. 7 is a front elevation view of the ball assembly if FIG. 2 showing a ball channel inlet;

FIG. 8 is a rear elevation view of the ball assembly of FIG. 1 showing a ball channel outlet; and

FIG. 9 is a schematic representation of an example of a respiration circuit system, the example of the system including a including the ball valve with a threaded connector attached to an gas inlet and a quick-connect fitting attached to the threaded connector as shown in FIG. 4 and further including a mask, nebulizer and connecting hoses.

### DETAILED DESCRIPTION

While various aspects and features of certain embodiments have been summarized above, the following detailed description illustrates a few embodiments in further detail to enable one of skill in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art, however, that other embodiments of the present invention may be practiced without some of these specific details. Several embodiments are described and claimed herein, and while various features are ascribed to different embodiments, it should be appreciated that the features described with respect to one embodiment may be incorporated with other embodiments as well. By the same token, however, no single feature or features of any described or claimed embodiment should be considered essential to every embodiment of the invention, as other embodiments of the invention may omit such features.

Unless otherwise indicated, all numbers used herein to express quantities, dimensions, and so forth used should be understood as being modified in all instances by the term “about.” In this application, the use of the singular includes the plural unless specifically stated otherwise, and use of the terms “and” and “or” means “and/or” unless otherwise indicated. Moreover, the use of the term “including,” as well as other forms, such as “includes” and “included,” should be considered non-exclusive. Also, terms such as “element” or “component” encompass both elements and components comprising one unit and elements and components that comprise more than one unit, unless specifically stated otherwise.

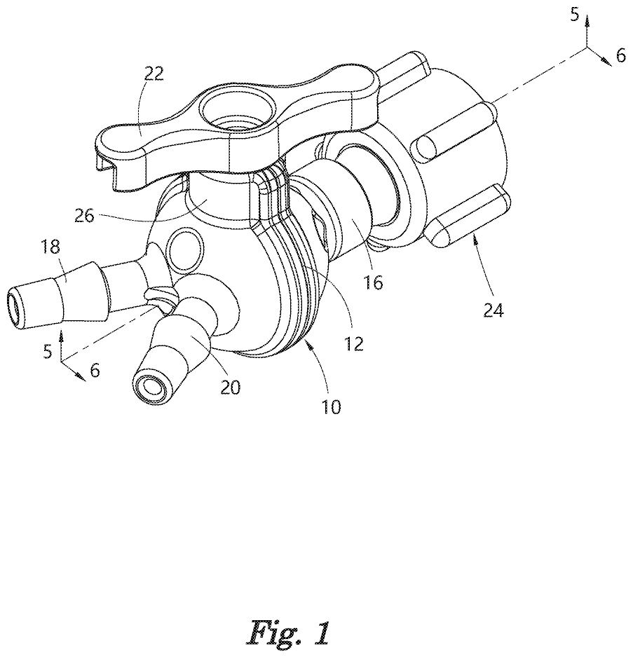

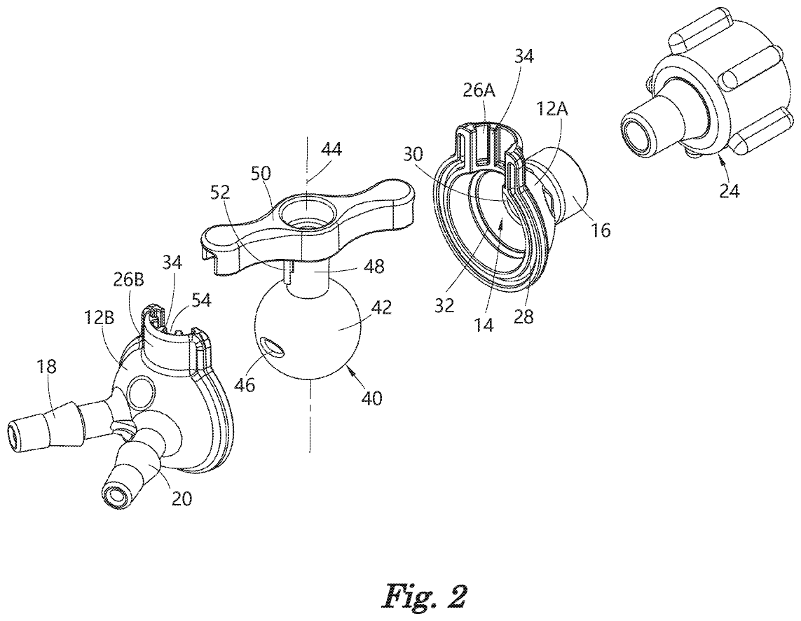

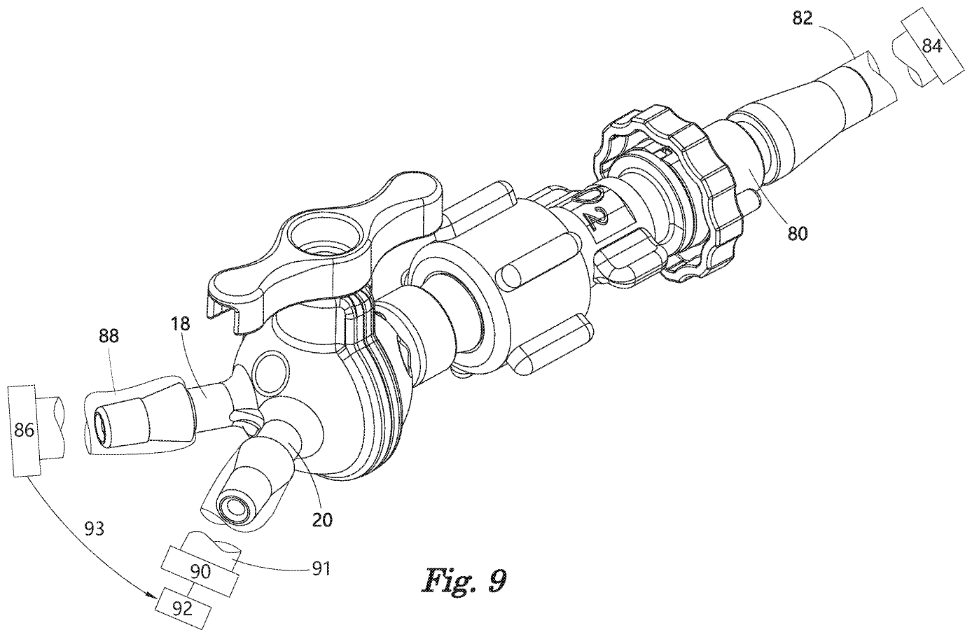

FIG. 1 shows a ball-valve 10 for use in a respiration circuit having a housing 12 defining a ball chamber 14 (see FIG. 2) in fluid communication with a gas inlet 16, a first barbed gas outlet 18 and second barbed gas outlet 20. A handle 22 protrudes from a top of the housing. In the illustrated embodiment, and optional threaded connector 24 is attached to the gas inlet 14 of the ball-valve 10 for connection to a pressurized gas supply (see FIG. 9).

The ball valve 10 is shown in greater detail in the exploded view of FIG. 2. In the illustrated embodiment, the housing 12 comprises an inlet side 12A and an outlet side 12B defining the ball chamber 14. The gas inlet 16 extends from the inlet side 12A and the first and second barbed gas outlets 18, 20 extend from the outlet side 12B substantially opposite the gas inlet 16. The inlet side 12A and the outlet side 12B further define a bearing collar 26 by mating of inlet side collar part 26A and outlet side collar part 26B. About the periphery of the inlet side 12A, the outlet side 12B, and inlet and outlet side collar parts 26A and 26B are mating flanges 28 for connection of the inlet and outlet sides 12A, 12B. Also visible in FIG. 2, within the ball chamber surface 30 are a number of channels 32 for receiving O-rings, as will be discussed below. Extending inwardly from the collar parts 26A and 26B are support flanges 34 that will be discussed in greater detail below.

A ball assembly 40 includes a ball 42 rotatable about an axis of rotation 44 when received in the ball chamber 14. A channel 46 extends through the ball 42 transverse to the axis of rotation 44; in some embodiments the channel extends perpendicular to the axis of rotation, in the manner illustrated herein. A stem 48 extends from the ball 42 along the axis of rotation 44 with a handle 50 attached to its distal end. As should be apparent from FIG. 2, the ball 42 is received in the ball chamber 14 with the stem 48 of the ball assembly 40 received in the collar 26. The handle 50 acts as an actuator for rotating the ball 40 about the axis of rotation 44. A protrusion 52 extends radially from the stem for engaging alignment detents 54 formed by the support flanges 34 extending radially inwardly from an inside surface of the collar 26.

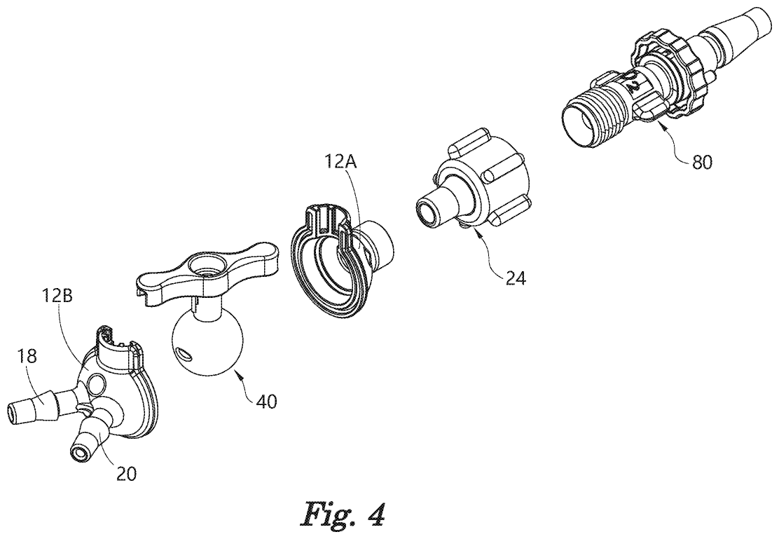

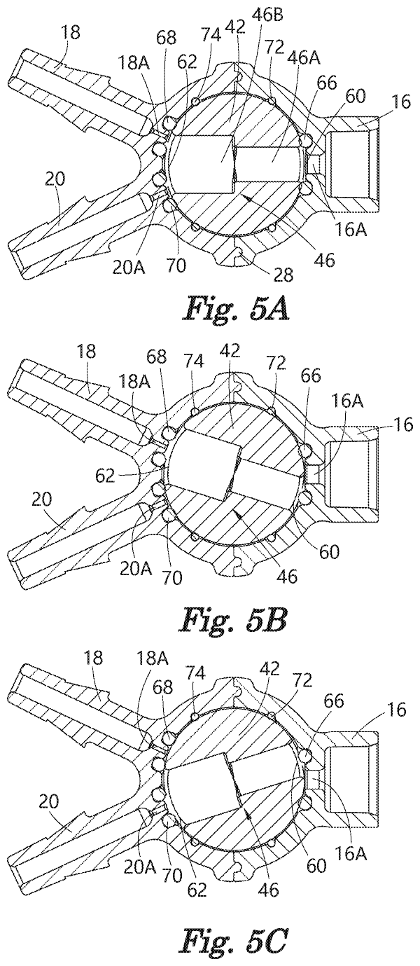

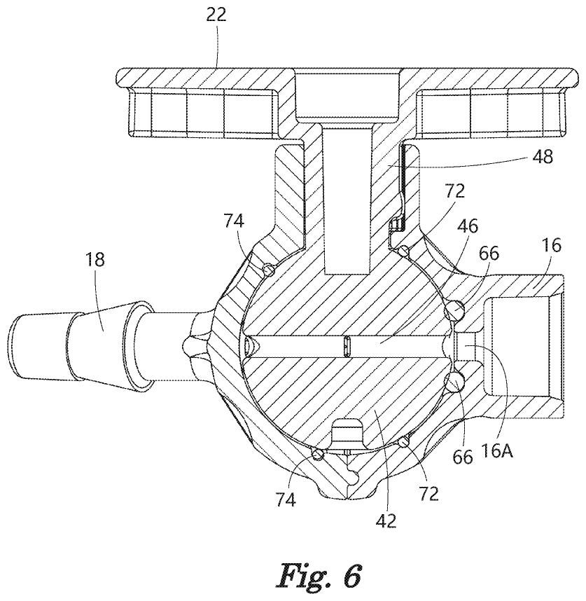

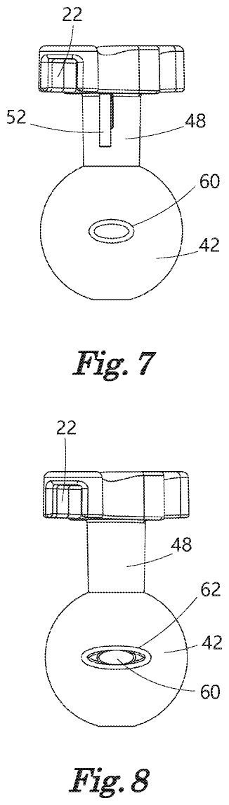

Referring to FIGS. 5A-D, the channel 46 has a small cross-section channel segment 46A terminating in a channel inlet 60 configured for selective fluid communication with the gas inlet 16, the small cross-section channel segment 46A being in fluid communication with a large cross-section channel segment 46B terminating in a channel outlet 62. In the illustrated embodiment, the channel has an elliptical cross-section (see FIGS. 2, 4, 7 and 8) with a major axis perpendicular to the axis of rotation 44 and a shorter minor axis parallel to the axis of rotation 44. The major axis in the small cross-section channel segment 46A is shorter than the major axis in the large cross-section channel segment 46B. Alternatively, the channel 46 may be provided with a more uniform transition between the relatively small channel inlet 60 and the relatively large channel outlet 62. Other alternative embodiments could have the cross-sectional segments 46A and 46B as well as the channel inlet 60 and the channel outlet 62 being of the same size and configuration. In other words, in such embodiments the channel 46, the channel inlet 60 and the channel outlet 62 have a uniform cross section between and including the channel inlet 60 and the channel outlet 62.

A number of O-rings are provided between the surface 30 of the ball chamber 14 and the ball 42. In the illustrated embodiment, these O-rings reside in correspondingly shaped O-ring channels 32 formed in the surface 30 of the ball chamber 14. Referring to FIG. 5A, a first elastomeric O-ring 66 surrounds the gas inlet 16, more particularly, an inlet nozzle 16A; a second elastomeric O-ring 68 surrounds the first gas outlet 18, more particularly a first outlet nozzle 18A; a third elastomeric O-ring 70 surrounds a second gas outlet 20, more particularly the second outlet nozzle 20A; a fourth elastomeric O-ring 72 surrounds the first elastomeric O-ring 66 to prevent gas flow between the ball surface and the ball chamber surface 30 outside the circumference of the fourth elastomeric O-ring 72 in any of the positions discussed below; and a fifth elastomeric O-ring 74 surrounds the second and third elastomeric O-rings 68, 70 and each of the gas outlet nozzles 18A and 20A, positioned to prevent gas flow between the ball surface and the ball 42 and the chamber surface 30 outside the circumference of the fifth elastomeric O-ring 74 in any of the positions discussed below.

The various components described above (excepting the O-rings) can be made of any suitable plastic using injection molding, machining, or any other suitable process. Non-limiting examples of plastics include acetal and acrylonitrile butadiene styrene (ABS). The O-rings can be made of numerous elastomeric materials, including silicone or rubber compositions, and optionally can be coated with a non-stick material such as polytetrafluoroethylene (PTFE), commonly known by the brand name Teflon®, or a lubricant. The assembled ball valve shown in various figures is held together by known processes such as sonic welding or solvent bonding, and the assembly is facilitated by the mating flanges 28 (see FIGS. 5A-D).

Referring to FIGS. 5A-D, the ball valve 10 is configured to operate as follows. By turning the handle 50 the ball 42 is rotated or actuated to several different positions. In a first position shown in FIG. 5A, the channel inlet 60 is at least partially within a circumference of the first O-ring 66 and the channel outlet 62 is at least partially within a circumference of the second O-ring 68 and the third O-ring 70. In a second position shown in FIG. 5B, the channel inlet 60 is at least partially within the circumference of the first O-ring 66, the channel outlet 62 is at least partially within the circumference of the second O-ring 68, and the channel outlet 62 is completely outside the circumference of the third O-ring 70. In a third position illustrated in FIG. 5C, the channel inlet 60 is at least partially within the circumference of the first O-ring 66, the channel outlet 62 is at least partially within the circumference of the third O-ring 70 and channel outlet 62 is completely outside the circumference of the second O-ring 68. In a fourth position illustrated in FIG. 5D, the gas inlet 16 is not in fluid communication with the channel inlet 60 because the channel inlet 60 is completely outside the circumference of the first O-ring 66, preventing gasflow through ball valve 10. As shown in FIG. 5D, the channel outlet 62 is also completely outside the circumference of the second and third O-rings 68, 70.

Embodiments of the ball valve 10 can include the protrusion 52 from the stem 48 to function in cooperation with the placement of the parallel flanges 56 as detents to nest between adjacent parallel flanges to seat the valve in a select one of the different positions. Embodiments could also be configured to prevent rotation of the handle to a position such as the fourth position described above stopping flow through the valve, depending upon the application of the ball valve 10.

As illustrated in FIGS. 5A-D, the nozzle 18A of the first gas outlet 18 is of a smaller diameter than the nozzle 20A of the second gas outlet 20. The diameters can be chosen to provide a select lower flow rate from the first gas outlet than the second gas outlet. Alternatively, both nozzles 18A and 20A could have the same diameter to provide the same flow rate. In certain embodiments, a pressurized gas flow source communicating with the gas inlet 16 has a standard pressure and flow rate allowing the flow rate and pressure through the first and second gas outlets 18, 20 to be substantially calibrated for select applications, as will be illustrated in the Example below. It should also be noted that various embodiments could include more than two gas outlets and the components could be modified consistent with the operational principles discussed above to provide various combinations of flow from the various gas outlets at varying relative flow rates.

Embodiments could include the ball valve 10 being a disposable that would be useful for infection control in a clinical setting. For example, embodiments with calibrated hole sizes could make the use of reusable flow meters unnecessary, thus eliminating reusable flow meters a source of infection.

### EXAMPLE

The following example is provided for illustrative purposes only and is not intended to limit the scope of the invention.

### Example 1

FIG. 9 schematically represents an application of the ball valve 10 as part of a respiration circuit system. The ball valve 10 has a threaded connector 24 mated to the gas inlet 16. A quick-connect fitting 80 is attached to the threaded connector 24 and hose 82 runs between the quick-connect fitting 80 and a pressurized gas supply 84 to provide fluid communication therebetween. Embodiments could include the quick-connect fitting 80 attaching directly to the pressurized gas supply 84 without the hose 82. The first gas outlet 18 is in fluid communication with a nebulizer 86, for example, via hose 88. The second gas outlet 20 is in fluid communication with a flow generator 90 inlet, for example, via hose 91. The flow generator may in turn be in fluid communication with a breathing mask 92 which may be, for example, a CPAP mask. Various intermediate components such as a pressure regulator or sensors may be in fluid communication between the gas outlet 20 and the flow generator 90, but any such components are omitted for clarity. An output of the nebulizer 86 is in fluid communication with the breathing mask via hose 93. The combined output of the nebulizer 86 and the second gas outlet can be delivered to a patient by the breathing mask 92. Certain embodiments can have the outlet nozzles configured to provide a flow rate of 15 liters per minute directly to the flow generator 90 and 10 liters per minute to the nebulizer 86 with the pressurized gas at a standard 50 PSI.

Another application of a ball valve 10 in accordance with this disclosure is with a resuscitator bag that requires front end oxygenation as well as oxygen for an accumulator bag.

The description of the various embodiments has been presented for purposes of illustration and description, but is not intended to be exhaustive or limiting of the invention to the form disclosed. The scope of the present invention is limited only by the scope of the following claims. Many modifications and variations will be apparent to those of ordinary skill in the art. The embodiments described and shown in the figures were chosen and described in order to explain the principles of the invention, the practical application, and to enable others of ordinary skill in the art to understand the invention for various embodiments with various modifications as are suited to the particular use contemplated. All references cited herein are incorporated in their entirety by reference.

1-19. (canceled)

20. A ball valve for use in a respiration circuit, comprising:
a housing defining a ball chamber, the ball chamber communicating with a gas inlet, a first gas outlet and a second gas outlet;
a ball received in the ball chamber, the ball being rotatable about an axis of rotation within the ball chamber, the ball defining a channel extending transverse to the axis of rotation between a first end portion adjacent the gas inlet and a second end portion adjacent the first and second gas outlets; and
an actuator coupled to the ball and configured to rotate the ball about the axis of rotation among at least three discrete positions including:
a first position in which the channel places the gas inlet in fluid communication with the first gas outlet while the second gas outlet is sealed from the channel;
a second position in which the channel places the gas inlet in fluid communication with the second gas outlet while the first gas outlet is sealed from the channel; and
a third position in which the channel places the gas inlet in fluid communication with both the first gas outlet and the second gas outlet;
wherein the first gas outlet is configured to be coupled to a respiratory device, and the second gas outlet is configured to be coupled to a breathing-gas delivery device configured to provide a positive airway pressure to a patient.

21. A The ball valve of claim 20, wherein the channel has a non-circular cross-section along at least a portion of its length.

22. A The ball valve of claim 21, wherein the channel has an elliptical cross-section.

23. A The ball valve of claim 22, wherein the channel has a major axis perpendicular to the axis of rotation and a shorter minor axis parallel to the axis of rotation.

24. A The ball valve of claim 22, wherein the channel includes a reduced-area segment adjacent the gas inlet and an enlarged-area segment adjacent the first and second gas outlets, the enlarged-area segment having a greater cross-sectional area than the reduced-area segment.

25. A The ball valve of claim 24, wherein a length of the major axis in the reduced-area segment is less than a length of the major axis in the enlarged-area segment.

26. A The ball valve of claim 20, further comprising:
a first elastomeric O-ring surrounding the gas inlet between a surface of the ball chamber and a surface of the ball;
a second elastomeric O-ring surrounding the first gas outlet between the surface of the ball chamber and the surface of the ball; and
a third elastomeric O-ring surrounding the second gas outlet between the surface of the ball chamber and the surface of the ball;
wherein, in the third position, the channel overlaps both the second and third elastomeric O-rings so that the gas inlet is in fluid communication with both the first and second gas outlets.

27. A The ball valve of claim 26, wherein, in the first position, the channel overlaps the first and second elastomeric O-rings and is completely outside a circumference of the third elastomeric O-ring, and wherein, in the second position, the channel overlaps the first and third elastomeric O-rings and is completely outside a circumference of the second elastomeric O-ring.

28. A The ball valve of claim 26, further comprising:
a fourth elastomeric O-ring surrounding the first elastomeric O-ring and the gas inlet between the surface of the ball chamber and the surface of the ball; and
a fifth elastomeric O-ring surrounding the second and third elastomeric O-rings and both of the first and second gas outlets between the surface of the ball chamber and the surface of the ball;
wherein the fourth and fifth elastomeric O-rings are positioned to prevent gas flow between the ball surface and the ball chamber surface outside respective circumferences of the fourth and fifth elastomeric O-rings at each of the first, second and third positions.

29. A The ball valve of claim 20, wherein the actuator comprises a stem extending from the ball along the axis of rotation and a manually graspable handle at a distal end of the stem.

30. A The ball valve of claim 29, wherein the actuator is rotatable to a fourth position in which the channel is sealed from both the first and second gas outlets.

31. A The ball valve of claim 30, wherein, in the fourth position, the channel is completely outside a circumference of the first elastomeric O-ring so that the gas inlet is sealed from the channel.

32. A The ball valve of claim 20, wherein the first and second gas outlets are formed by respective outlet nozzles extending from a common exterior surface of the housing that faces away from the gas inlet.

33. A The ball valve of claim 20, wherein the first gas outlet has a lower nominal flow capacity than the second gas outlet under a common upstream gas pressure.

34. A The ball valve of claim 20, wherein the gas inlet comprises a threaded connector configured to receive a removable quick-connect fitting for coupling the ball valve to the common gas source.

35. A The ball valve of claim 20, wherein the housing comprises an inlet side and an outlet side that are permanently joined around a circumferential flange to enclose the ball.

36. A The ball valve of claim 20, wherein the ball, the housing and the actuator are formed of an injection-molded thermoplastic material selected from the group consisting of acetal and acrylonitrile-butadiene-styrene.

37. A The ball valve of claim 20, wherein the respiratory device is a nebulizer.

38. A A method of delivering pressurized breathable gas and medication to a patient from a common gas source, the method comprising:
coupling a gas inlet of a multi-position ball valve to the common gas source;
coupling a first gas outlet of the ball valve to an inlet of a respiratory device;
coupling a second gas outlet of the ball valve to an inlet of a breathing-gas delivery device configured to provide a positive airway pressure to the patient, wherein a respiratory device outlet being in fluid communication with the breathing-gas delivery device;
rotating a ball of the ball valve to a first operating position in which a channel within the ball places the gas inlet in fluid communication with both the first gas outlet and the second gas outlet so that the common gas source simultaneously drives the respiratory device and the breathing-gas delivery device; and
applying the breathing-gas delivery device to the patient while the ball is in the first operating position to concurrently deliver positive-pressure breathing gas and medication to the patient.

39. A The method of claim 38, further comprising, after concurrently delivering positive-pressure breathing gas and medication, rotating the ball to a second operating position in which the gas inlet is in fluid communication with the second gas outlet and sealed from the first gas outlet, thereby delivering positive-pressure breathing gas to the patient without operating the respiratory device.

40. A The method of claim 38, further comprising, before rotating the ball to the first operating position, rotating the ball to a third operating position in which the gas inlet is in fluid communication with the first gas outlet and sealed from the second gas outlet, thereby operating the nebulizer without operating the breathing-gas delivery device.

41. A The method of claim 38, further comprising rotating the ball to a shut-off position in which the gas inlet is sealed from both the first and second gas outlets to interrupt gas flow through the ball valve without disconnecting the respiratory device or the breathing-gas delivery device.

42. A The method of claim 38, wherein the breathing-gas delivery device comprises a continuous positive airway pressure (CPAP) device.

43. A The method of claim 38, wherein, when the ball is in the first operating position and the common gas source is at about 50 psi, the respiratory device is driven at 10 liters per minute and the breathing-gas delivery device is driven at 15 liters per minute.

44. A The method of claim 38, wherein the channel in the ball has an elliptical cross-section.

45. A The method of claim 38, further comprising sensing a rotational position of the ball by engagement of a projection on a stem of the ball with discrete detent surfaces on the housing corresponding to the first, second and third operating positions.

46. A The method of claim 38, wherein the respiratory device delivers an aerosolized medicament into a breathing volume defined by the breathing-gas delivery device.

47. A The method of claim 38, wherein the respiratory device and the breathing-gas delivery device remain connected to the ball valve while the ball is rotated among the operating positions.

48. A The method of claim 38, wherein the respiratory device is a nebulizer.
